(19) United States
(12) Patent Application Publication
(10) Pub. No.: US 2020/0407290 A1
(43) Pub. Date: Dec. 31, 2020

(54) PROCESS FOR TRANSALKYLATING BENZENE

(71) Applicant: UOP LLC, Des Plaines, IL (US)

(72) Inventors: Stanley Joseph Frey, Palatine, IL (US); Gavin Paul Towler, Inverness, IL (US); Rajeev Gautam, Kildeer, IL (US)

(21) Appl. No.: 16/456,857

(22) Filed: Jun. 28, 2019

(51) Int. Cl. C07C 6/12; C10G 47/00; C10L 1/06; C10G 47/36

(52) U.S. Cl. CPC C07C 6/12; C10G 47/00; C10L 1/06; C10L 2270/023; C10L 2290/24; C10L 2200/0423; C10G 47/36

(57) ABSTRACT

Benzene is transalkylated with existing aromatic alkyl groups in a hydrocracking reactor by feeding a benzene stream and alkylated aromatic compounds to a hydrocracking unit. Alkyl groups migrate from heavier alkylated aromatic compounds to the benzene compounds during transalkylation in the hydrocracking unit. The hydrocracking conditions do not prevent the alkyl groups from transferring from one benzene ring to another benzene ring.

### FIELD

The field is hydrocracking and transalkylating hydrocarbon streams.

### BACKGROUND

Benzene has come under scrutiny after its identification as a carcinogen. Benzene may be used for petrochemical production, but oil refineries are loathe to produce more benzene than is needed for petrochemical use. Oil refineries are required to comply with regulatory limits on the concentration of benzene allowed in gasoline. Benzene precursors such as methylcyclopentane and cyclohexane are rerouted from reformers to avoid unwanted production of excess benzene.

Presently there are three primary ways to deal with refinery benzene. The first is to hydrogenate it to naphthene in a hydrogenation unit. However, this reduces the gasoline pool octane, increases gasoline pool Reid vapor pressure, consumes hydrogen and requires construction of a dedicated benzene hydrotreating unit. Another method is to alkylate benzene over an acid catalyst with a light olefin to make a more acceptable, alkylated benzene. Alkylbenzene is not subject to the same regulatory restrictions as benzene. This option also requires a dedicated benzene alkylation unit to be built and sufficient availability of a suitable light olefin stream. Finally, the benzene can be isolated and exported for use to a petrochemical facility. This solution, however, requires the refiner to identify a willing petrochemical customer, purify benzene to meet a minimal specification, additional terminal facilities, and additional difficulties presented with the production of an additional product.

Hydrocracking can include processes which convert hydrocarbons in the presence of hydrocracking catalyst and hydrogen to more valuable products. Hydrocracking is a hydroprocessing process in which hydrocarbons crack in the presence of hydrogen and hydrocracking catalyst to lower molecular weight hydrocarbons. Depending on the desired output, a hydrocracking unit may contain one or more beds of the same or different catalyst. Hydrocracking can be performed with one or two hydrocracking reactor stages. Typically, naphtha range streams are not sent to a hydrocracker as naphtha is a target product from a hydrocracker, not a feed. Hydrotreating is a hydroprocessing process used to remove heteroatoms such as sulfur and nitrogen from hydrocarbon streams to meet fuel specifications and to saturate olefinic compounds. Hydrotreating can be performed at high or low pressures but is typically operated at lower pressure than hydrocracking.

Transalkylation is a process in which benzene and/or toluene are reacted with C₈+ aromatics to form more methylated aromatics. Transalkylation is performed with an acidic catalyst on a feed stream composed primarily of monoaromatic naphtha boiling range; i.e., less than 390° C. material. Transalkylation is performed in the absence of hydrocracking reactions, except for mild aromatics dealkylation, at moderate temperature and pressure, to avoid loss of desirable aromatic rings and the production of undesirable dry gas. Transalkylation is primarily practiced in petrochemical aromatic production facilities on hydrotreated, highly aromatic streams to convert toluene and C₉+ aromatics to benzene and xylenes.

As refinery streams with high concentrations of sulfur and nitrogen compounds and significant non-aromatic concentrations in the naphtha boiling range are typically not hydrocracked or transalkylated to produce naphtha, there is a continuing need, therefore, for improved methods for reducing benzene production in oil refineries.

### BRIEF SUMMARY

We have found that benzene may be readily transalkylated with alkyl groups from heavier alkylated aromatics in a hydrocracking reactor by feeding a benzene stream and alkylated aromatic compounds to a hydrocracking unit. Alkyl groups tend to migrate from alkylated aromatic compounds to the benzene compounds during concurrent transalkylation in the hydrocracking unit. We have found a process in which alkyl groups transfer from heavier aromatics to benzene such that the concurrent hydrocracking reaction does not prevent the alkyl transfer or cause excessive loss of naphtha from the feed.

### BRIEF DESCRIPTION OF THE DRAWINGS

FIG. 1 is a simplified process flow diagram.

FIG. 2 is an alternative process flow diagram to FIG. 1.

FIG. 3 is an additional process flow diagram to FIGS. 1 and 2.

### DEFINITIONS

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without passing through a fractionation or conversion unit to undergo a compositional change due to physical fractionation or chemical conversion.

The term “bypass” means that the object is out of downstream communication with a bypassing subject at least to the extent of bypassing.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns omit a reboiler at a bottom of the column and instead provide heating requirements and separation impetus from a fluidized inert media such as steam.

As used herein, the term “True Boiling Point” (TBP) means a test method for determining the boiling point of a material which corresponds to ASTM D2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5” or “T95” means the temperature at which 5 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “conversion” means conversion of feed to material that boils at or below the diesel boiling range. The diesel cut point of the diesel boiling range is between about 343° and about 399° C. (650° to 750° F.) using the True Boiling Point distillation method.

As used herein, the term “diesel boiling range” means hydrocarbons boiling in the range of between about 132° and about 399° C. (270° to 750° F.) using the True Boiling Point distillation method.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant”, “predominantly” or “predominate” means greater than 50%, suitably greater than 75% and preferably greater than 90%.

### DETAILED DESCRIPTION

We have discovered that when conditions are maintained for high cracking and low hydrogenation benzene can be transalkylated with alkyls from alkylated aromatic compounds in the feedstock to produce naphtha boiling range alkylbenzene in a hydrocracking reactor causing a net increase in total naphtha range aromatics. Transalkylation of benzene over an acid function catalyst with another multiple alkyl-substituted aromatic results in two alkyl substituted aromatics. This method of benzene conversion increases the gasoline pool volume, shifts diesel boiling fraction polyalkylated aromatics to the gasoline boiling fraction thereby increasing gasoline octane-barrels and increasing diesel fraction cetane. Transalkylation does not consume hydrogen or negatively affect octane rating. The benzene stream and the polyalkylated aromatic stream do not need to be highly purified. Transalkylation can be conducted in any environment conducive to transalkylation such as in a low severity hydrocracking unit. To effectively yield maximum naphtha octane it is also advantageous to operate in a regime of low hydrogenation to maximize aromatic yield.

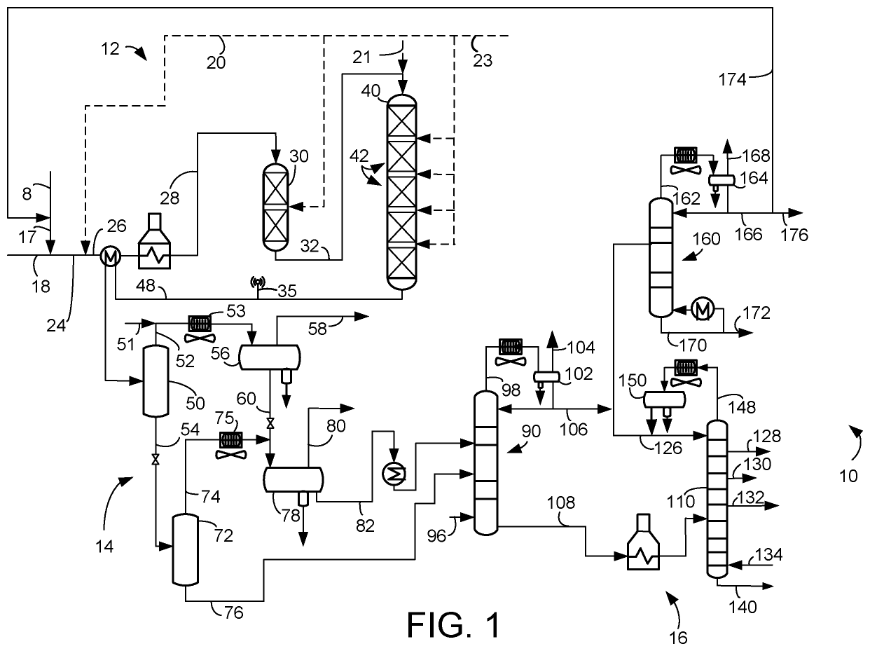

In FIG. 1, a hydrocracking unit 10 for hydrocracking hydrocarbons comprises a suitable environment for transalkylation of benzene to alkylaromatics. The hydrocracking unit 10 comprises a hydrocracking reactor section 12, a separation section 14, and a fractionation section 16. A hydrocarbonaceous distillate feed stream in a distillate line 18 and a hydrogen-rich stream in hydrogen line 20 are fed to the hydrocracking reactor section 12. Hydrocracked effluent is separated in the fractionation section 16.

In the hydrocracking reactor section 12 hydrocracking of hydrocarbons occurs. Hydrocracking refers to a process in which hydrocarbons crack in the presence of hydrogen to lower molecular weight hydrocarbons.

In one aspect, the process and apparatus described herein are particularly useful for hydrocracking a distillate feed stream in a distillate feed line 18 conventionally fed to a hydrocracking unit comprising a distillate fraction feedstock. Illustrative distillate fraction feedstocks conventional for hydrocracking units include a distillate fraction stream having an initial boiling point (IBP) above at least about 193° C. (380° F.), suitably at least about 199° C. (390° F.), such as diesel boiling range streams. Other conventional distillate fraction feedstocks may be heavier hydrocarbon streams having an IBP above at least about 288° C. (550° F.), such as atmospheric gas oils, vacuum gas oil (VGO) having T5 and/or T95 between about 315° C. (600° F.) and about 650° C. (1200° F.), deasphalted oil, coker distillates, straight run distillates, pyrolysis-derived oils, high boiling synthetic oils, cycle oils, clarified slurry oils, shale oil and catalytic cracker distillates. The conventional distillate feed stream will include substantial concentration of alkylated aromatic compounds.

An unconventional feed stream fed to the hydrocracking unit 10 in a naphtha feed line 17 comprises a naphtha fraction stream having an IBP and/or a T5 between about 40 and about 80° C. and a T95 and/or an end point between about 80 and about 210° C. and preferably no more than about 180° C. Naphtha is not typically sent to a hydrocracking unit because benzene does not typically crack and the naphtha that does crack makes dry gas and liquefied petroleum gas which are less valuable than naphtha.

The naphtha fraction stream may comprise at least about 5 wt % benzene and suitably at least about 10 wt % benzene or about 10 wt % benzene and toluene. More suitably, the naphtha fraction stream may comprise at least about 20 wt % benzene or 20 wt % benzene and toluene. Most suitably, the naphtha fraction stream may comprise at least about 30 wt % benzene or 30 wt % benzene and toluene. Preferably, the naphtha fraction stream may comprise at least about 40 wt % benzene or about 40 wt % benzene and toluene. More preferably, the naphtha fraction stream may comprise at least about 50 wt % benzene or about 50 wt % benzene and toluene. Most preferably, the naphtha fraction stream may comprise at least about 60 wt % benzene or about 60 wt % benzene and toluene. The toluene, xylenes, and trimethylbenzene behave similarly to benzene in the receipt of alkyl groups in the process. However, toluene, xylenes, and trimethylbenzene may also contribute alkyl groups to benzene or to other alkylaromatics.

The naphtha fraction stream in naphtha feed line 17 may comprise a naphtha recycle stream in a recycle line 174 supplementing a fresh naphtha stream in a fresh line 8. The fresh naphtha stream may comprise straight run naphtha from a crude column side cut or FCC naphtha such as from a debutanizer column bottoms which in either case may be split to remove the heavy naphtha to provide the benzene-rich light naphtha for hydrocracking. The naphtha fraction stream in naphtha feed line 17 may be mixed with the distillate fraction in the distillate feed line 18 and fed together as a hydrocarbon feed stream in a hydrocarbon feed line 24 to the hydrocracking reactor 40. Alternatively, the naphtha fraction stream and the distillate fraction stream may be fed to the hydrocracking reactor 40 separately.

One of the naphtha fraction stream and the distillate fraction stream may also comprise alkylated aromatic compounds and are preferably polyalkylated aromatic compounds. The alkylated aromatic compounds may have one or more aromatic rings. The alkylated aromatic compounds are necessary to provide the alkyl groups for transalkylation of benzene. The alkylated aromatic compounds will typically be in the distillate fraction stream. Preferably, at least twice as many moles of alkyl groups on aromatic compounds are fed to the hydrocracking reactor 40 as moles of benzene molecules.

The hydrocracking reactor section 12 may optionally include a pre-hydrotreating reactor 30. Hydrotreating is a process wherein hydrogen is contacted with hydrocarbon in the presence of suitable catalysts which are primarily active for the removal of heteroatoms, such as sulfur, nitrogen and metals from the hydrocarbon feedstock. In hydrotreating, hydrocarbons with double and triple bonds may be saturated. The cloud point of the hydrotreated product may also be reduced. The subject process and apparatus will be described with the hydrocracking reactor section 12 including a hydrotreating reactor 30 and a hydrocracking reactor 40.

The hydrogen stream in the hydrogen line 20 may split off from a hydrocracking hydrogen line 23. The hydrogen stream in line 20 may be a hydrotreating hydrogen stream. The hydrotreating hydrogen stream may join the hydrocarbon stream in the hydrocarbon line 24 to provide a hydrogen-mixed, hydrocarbon feed stream in a hydrocarbon feed line 26. The hydrogen-mixed, hydrocarbon feed stream in the hydrocarbon feed line 26 may be heated by heat exchange with a hydrocracked stream in a hydrocracked line 48 and in a fired heater. The heated hydrocarbon feed stream in line 28 may be fed to the hydrotreating reactor 30.

The hydrotreating reactor 30 may be a fixed bed reactor that comprises one or more vessels, single or multiple beds of catalyst in each vessel, and various combinations of hydrotreating catalyst in one or more vessels.

The hydrotreating reactor 30 may comprise a guard bed of specialized material for pressure drop mitigation followed by one or more beds of higher quality hydrotreating catalyst. The guard bed filters particulates and picks up contaminants in the hydrocarbon feed stream such as metals like nickel, vanadium, silicon and arsenic which deactivate the catalyst. The guard bed may comprise material similar to the hydrotreating catalyst. Supplemental hydrogen may be added at an interbed location between catalyst beds in the hydrotreating reactor 30.

Suitable hydrotreating catalysts are any known conventional hydrotreating catalysts and include those which are comprised of at least one Group VIII metal, preferably iron, cobalt and nickel, more preferably cobalt and/or nickel and at least one Group VI metal, preferably molybdenum and tungsten, on a high surface area support material, preferably alumina. Other suitable hydrotreating catalysts include zeolitic catalysts, as well as noble metal catalysts where the noble metal is selected from palladium and platinum. It is within the scope of the present description that more than one type of hydrotreating catalyst be used in the same hydrotreating reactor 30. The Group VIII metal is typically present in an amount ranging from about 2 to about 20 wt %, preferably from about 4 to about 12 wt %. The Group VI metal will typically be present in an amount ranging from about 1 to about 25 wt %, preferably from about 2 to about 25 wt %.

Preferred hydrotreating reaction conditions include a temperature from about 290° C. (550° F.) to about 455° C. (850° F.), suitably 316° C. (600° F.) to about 427° C. (800° F.) and preferably 343° C. (650° F.) to about 399° C. (750° F.), a pressure from about 2.8 MPa (gauge) (400 psig) to about 17.5 MPa (gauge) (2500 psig), a liquid hourly space velocity of the fresh hydrocarbonaceous feedstock from about 0.1 hr⁻¹, suitably from about 0.5 hr⁻¹, to about 5 hr⁻¹, preferably from about 1.5 to about 4 hr⁻¹, and a hydrogen rate of about 84 Nm³/m³ (500 scf/bbl), to about 1,011 Nm³/m³ oil (6,000 scf/bbl), preferably about 168 Nm³/m³ oil (1,000 scf/bbl) to about 1,250 Nm³/m³ oil (7,500 scf/bbl), with a hydrotreating catalyst or a combination of hydrotreating catalysts.

The hydrocarbon feed stream in the hydrocarbon feed line 28 may be hydrotreated with the hydrotreating hydrogen stream from hydrotreating hydrogen line 20 over the hydrotreating catalyst in the hydrotreating reactor 30 to provide a hydrotreated hydrocarbon stream that exits the hydrotreating reactor 30 in a hydrotreated hydrocarbon feed line 32.

The hydrotreated hydrocarbon feed stream may be fed directly to the hydrocracking reactor 40 without separation. The hydrotreated hydrocarbon feed stream may be mixed with a hydrocracking hydrogen stream in a hydrocracking hydrogen line 21 taken from the hydrocracking hydrogen line 23 and be fed through an inlet to the hydrocracking reactor 40 to be hydrocracked. The hydrocarbon feed stream may comprise at least about 30 wppm nitrogen and at least about 0.1 wt % sulfur

Hydrocracking is a process in which hydrocarbons crack in the presence of hydrogen to lower molecular weight hydrocarbons. The hydrocracking reactor 40 may be a fixed bed reactor that comprises one or more vessels, single or multiple catalyst beds 42 in each vessel, and various combinations of hydrotreating catalyst and/or hydrocracking catalyst in one or more vessels. The hydrocracking reactor 40 may also be operated in a conventional continuous gas phase, a moving bed or a fluidized bed hydrocracking reactor.

The hydrocracking reactor 40 comprises a plurality of hydrocracking catalyst beds 42. If the hydrocracking reactor section 12 does not include a hydrotreating reactor 30, the catalyst beds 42 in the hydrocracking reactor 40 may include a hydrotreating catalyst for the purpose of saturating, demetallizing, desulfurizing or denitrogenating the hydrocarbon feed stream before it is hydrocracked with the hydrocracking catalyst in subsequent vessels or catalyst beds 42 in the hydrocracking reactor 40.

The hydrotreated hydrocarbon feed stream is hydrocracked over a hydrocracking catalyst in a hydrocracking reactor in the presence of a hydrocracking hydrogen stream from a hydrocracking hydrogen line 21 to provide a hydrocracked effluent stream. Specifically, the distillate fraction in the hydrocarbon feed stream in the hydrotreated hydrocarbon feed line 32 may be hydrocracked in the hydrocracking reactor 40 with the hydrogen stream over hydrocracking catalyst to provide a hydrocracked stream. Additionally, benzene in the naphtha fraction is transalkylated with alkyl groups from the alkylated aromatic compounds to produce alkylbenzene.

A hydrogen manifold may deliver supplemental hydrogen streams to one, some or each of the catalyst beds 42. In an aspect, the supplemental hydrogen is added to each of the hydrocracking catalyst beds 42 at an interbed location between adjacent beds, so supplemental hydrogen is mixed with hydrocracked effluent exiting from the upstream catalyst bed 42 before entering the downstream catalyst bed 42.

At least about 5 wt % of the distillate fraction boiling in the range of at least about 193° C. (380° F.), suitably at least about 199° C. (390° F.), is converted to material boiling below about 193° C. (380° F.), suitably at least about 199° C. (390° F.). Suitably, at least about 10 wt % of the distillate fraction boiling in the range of at least about 193° C. (380° F.), suitably at least about 199° C. (390° F.), is converted to material boiling below about 193° C. (380° F.), suitably at least about 199° C. (390° F.). More suitably, at least about 15 wt % of the distillate fraction boiling in the range of at least about 193° C. (380° F.), suitably at least about 199° C. (390° F.), is converted to material boiling below about 193° C. (380° F.), suitably at least about 199° C. (390° F.). Most suitably, at least about 20 wt % of the distillate fraction boiling in the range of at least about 193° C. (380° F.), suitably at least about 199° C. (390° F.), is converted to material boiling below about 193° C. (380° F.), suitably at least about 199° C. (390° F.). Preferably, at least about 25 wt % of the distillate fraction boiling in the range of at least about 193° C. (380° F.), suitably at least about 199° C. (390° F.), is converted to material boiling below about 193° C. (380° F.), suitably at least about 199° C. (390° F.). More preferably, at least about 30 wt % of the distillate fraction boiling in the range of at least about 193° C. (380° F.), suitably at least about 199° C. (390° F.), is converted to material boiling below about 193° C. (380° F.), suitably at least about 199° C. (390° F.). Most preferably, at least about 35 wt % of the distillate fraction boiling in the range of at least about 193° C. (380° F.), suitably at least about 199° C. (390° F.), is converted to material boiling below about 193° C. (380° F.), suitably at least about 199° C. (390° F.).

The hydrocracking reactor may provide a total conversion of at least about 20 vol % and typically greater than about 60 vol % of the hydrocarbon stream in the hydrocarbon line 32 to products boiling below the cut point of the heaviest desired product which is typically diesel. The hydrocracking reactor 40 may operate at partial conversion of more than about 30 vol % or full conversion of at least about 90 vol % of the feed based on total conversion. The hydrocracking reactor 40 may be operated at mild hydrocracking conditions which will provide about 20 to about 60 vol %, preferably about 20 to about 50 vol %, total conversion of the hydrocarbon feed stream to product boiling below the diesel cut point.

The hydrocracking catalyst may utilize an acidic amorphous silica-alumina base or an acidic zeolite cracking base combined with one or more Group VIII or Group VIB metal hydrogenating components. Additional hydrogenating components may be selected from Group VIB for incorporation with the zeolite base.

The zeolite cracking bases are sometimes referred to in the art as molecular sieves and are usually composed of silica, alumina and one or more exchangeable cations such as sodium, magnesium, calcium, rare earth metals, etc. They are further characterized by crystal pores of relatively uniform diameter between about 4 and about 14 Angstroms. It is preferred to employ zeolites having a relatively high silica/alumina mole ratio between about 3 and about 12. Suitable zeolites found in nature include, for example, mordenite, stilbite, heulandite, ferrierite, dachiardite, chabazite, erionite and faujasite. Suitable synthetic zeolites include, for example, the B, X, Y and L crystal types, e.g., synthetic faujasite and mordenite. The preferred zeolites are those having crystal pore diameters between about 8 and 12 Angstroms, wherein the silica/alumina mole ratio is about 4 to 6. One example of a zeolite falling in the preferred group is synthetic Y molecular sieve.

The natural occurring zeolites are normally found in a sodium form, an alkaline earth metal form, or mixed forms. The synthetic zeolites are nearly always prepared in the sodium form. In any case, for use as a cracking base it is preferred that most or all of the original zeolitic monovalent metals be ion-exchanged with a polyvalent metal and/or with an ammonium salt followed by heating to decompose the ammonium ions associated with the zeolite, leaving in their place hydrogen ions and/or exchange sites which have actually been decationized by further removal of water. Hydrogen or “decationized” Y zeolites of this nature are more particularly described in U.S. Pat. No. 3,100,006.

Mixed polyvalent metal-hydrogen zeolites may be prepared by ion-exchanging with an ammonium salt, then partially back exchanging with a polyvalent metal salt and then calcining. In some cases, as in the case of synthetic mordenite, the hydrogen forms can be prepared by direct acid treatment of the alkali metal zeolites. In one aspect, the preferred cracking bases are those which are at least about 10 wt %, and preferably at least about 20 wt %, metal-cation-deficient, based on the initial ion-exchange capacity. In another aspect, a desirable and stable class of zeolites is one wherein at least about 20 wt % of the ion exchange capacity is satisfied by hydrogen ions.

The active metals employed in the preferred hydrocracking catalysts of the present invention as hydrogenation components are those of Group VIII, i.e., iron, cobalt, nickel, ruthenium, rhodium, palladium, osmium, iridium and platinum. In addition to these metals, other promoters may also be employed in conjunction therewith, including the metals of Group VIB, e.g., molybdenum and tungsten. The amount of hydrogenating metal in the catalyst can vary within wide ranges. Broadly speaking, any amount between about 0.05 wt % and about 30 wt % may be used. In the case of the noble metals, it is normally preferred to use about 0.05 to about 2 wt % noble metal. Typically, the hydrogenating metal in the catalyst is greater than about 1 wt %; suitably, at least about 2 wt %; and preferably, at least about 3 wt %.

The method for incorporating the hydrogenation metal is to contact the base material with an aqueous solution of a suitable compound of the desired metal wherein the metal is present in a cationic form. Following addition of the selected hydrogenation metal or metals, the resulting catalyst powder is then filtered, dried, pelleted with added lubricants, binders or the like if desired, and calcined in air at temperatures of, e.g., about 371° C. (700° F.) to about 648° C. (200° F.) in order to activate the catalyst and decompose ammonium ions. Alternatively, the base component may be pelleted, followed by the addition of the hydrogenation component and activation by calcining.

The foregoing catalysts may be employed in undiluted form, or the powdered catalyst may be mixed and copelleted with other relatively less active catalysts, diluents or binders such as alumina, silica gel, silica-alumina cogels, activated clays and the like in proportions ranging between about 5 and about 90 wt %. These diluents may be employed as such or they may contain a minor proportion of an added hydrogenating metal such as a Group VIB and/or Group VIII metal. Additional metal promoted hydrocracking catalysts may also be utilized in the process of the present invention which comprises, for example, aluminophosphate molecular sieves, crystalline chromosilicates and other crystalline silicates. Crystalline chromosilicates are more fully described in U.S. Pat. No. 4,363,178.

By one approach, the hydrocracking conditions may include a temperature from about 290° C. (550° F.) to about 468° C. (875° F.), suitably at least about 300° C. (572° F.), and preferably about 343° C. (650° F.) to about 445° C. (833° F.), a pressure of at least about 3.5 MPa (500 psig), and suitably from about 4.8 MPa (gauge) (700 psig) to about 20.7 MPa (gauge) (3000 psig), a liquid hourly space velocity (LHSV) from about 0.4 to less than about 2.5 hr⁻¹ and a hydrogen rate of about 421 Nm³/m³ (2,500 scf/bbl) to about 2,527 Nm³/m³ oil (15,000 scf/bbl). Preferably, high cracking and low hydrogenation conditions are achieved by maintaining the pressure at about 4.83 MPag (700 psig) to about 10.34 MPag (1500 psig) to achieve a high naphtha octane rating and to avoid aromatic hydrogenation. The hydrocracked stream may exit the hydrocracking reactor 40 in the hydrocracked line 48. The hydrocracked stream comprises a higher concentration of alkylbenzene and a lower concentration of distillate boiling in the range of at least about 193° C. (380° F.), suitably at least about 199° C. (390° F.), than the hydrocarbon feed stream.

The hydrocracked stream may be separated in the separation section 14 in downstream communication with the hydrocracking reactor 40. The separation section 14 comprises one or more separators in downstream communication with the hydrocracking reactor comprising the hydrotreating reactor 30 and/or the hydrocracking reactor 40. The hydrocracked effluent stream in the hydrocracked line 48 may in an aspect be heat exchanged with the hydrocarbon feed stream in the hydrocarbon feed line 26 and be delivered to a hot separator 50.

The hot separator 50 separates the hydrocracked effluent stream to provide a hydrocarbonaceous, hot vapor stream in a hot overhead line 52 extending from a top of the hot separator 50 and a hydrocarbonaceous, hot liquid stream in a hot bottoms line 54 extending from a bottom of the hot separator 50. The hot separator 50 may be in downstream communication with the hydrocracking reactor comprising the hydrotreating reactor 30 and/or the hydrocracking reactor 40. The hot separator 50 operates at about 77° C. (350° F.) to about 371° C. (700° F.) and preferably operates at about 232° C. (450° F.) to about 315° C. (600° F.). The hot separator 50 may be operated at a slightly lower pressure than the hydrocracking reactor 40 accounting for pressure drop through intervening equipment. The hot separator 50 may be operated at pressures between about 3.4 MPa (gauge) (493 psig) and about 20.4 MPa (gauge) (2960 psig). The hydrocarbonaceous, hot vapor stream taken in the hot overhead line 52 may have a temperature of the operating temperature of the hot separator 50.

The hot vapor stream in the hot overhead line 52 may be cooled with an air cooler 53 before entering a cold separator 56. To prevent deposition of salts in the hot overhead line 52 transporting the hot vapor stream, a suitable amount of wash water may be introduced into the hot overhead line 52 upstream of the air cooler 53 by water line 51 at a point in the hot overhead line.

The hot vapor stream may be separated in the cold separator 56 to provide a cold vapor stream comprising a hydrogen-rich gas stream in a cold overhead line 58 extending from a top of the cold separator 56 and a cold liquid stream in a cold bottoms line 60 extending from a bottom of the cold separator 56. The cold separator 56 serves to separate hydrogen rich gas from hydrocarbon liquid in the hydrocracked stream for recycle to the reactor section 12 in the cold overhead line 58. The cold separator 56 may be operated at about 100° F. (38° C.) to about 150° F. (66° C.), suitably about 115° F. (46° C.) to about 145° F. (63° C.), and just below the pressure of the hydrocracking reactor 40 and the hot separator 50 accounting for pressure drop through intervening equipment. The cold separator 56 may be operated at pressures between about 3 MPa (gauge) (435 psig) and about 20 MPa (gauge) (2,900 psig). The cold separator 56 may also have a boot for collecting an aqueous phase. The cold liquid stream in the cold bottoms line 60 may have a temperature of the operating temperature of the cold separator 56.

The hydrocarbonaceous hot liquid stream in the hot bottoms line 54 may be let down in pressure and separated in a hot flash drum 72 to provide a hot flash vapor stream of light ends and hydrogen in a hot flash overhead line 74 extending from a top of the hot flash drum and a hot flash liquid stream in a hot flash bottoms line 76 extending from a bottom of the hot flash drum. A product stripping column 90 may be in direct, downstream communication with the hot flash drum 72 and the hot flash bottoms line 76. The hot flash drum 72 may be operated at the same temperature as the hot separator 50 but at a lower pressure of between about 1.4 MPa (gauge) (200 psig) and about 6.9 MPa (gauge) (1000 psig), suitably no more than about 3.8 MPa (gauge) (550 psig).

In an aspect, the cold liquid stream in the cold bottoms line 60 may be let down in pressure and flashed in a cold flash drum 78 to separate the cold liquid stream in the cold bottoms line 60. The cold flash drum 78 may separate the cold liquid stream in the cold bottoms line 60 to provide a cold flash vapor stream in a cold flash overhead line 80 extending from a top of the cold flash drum 78 and a cold flash liquid stream in a cold flash bottoms line 82 extending from a bottom of the cold flash drum. In an aspect, light gases such as hydrogen sulfide may be stripped from the cold flash liquid stream in the cold flash bottoms line 82.

The cold flash drum 78 may be in downstream communication with the cold bottoms line 60 of the cold separator 56. The cold flash drum 78 may be operated at the same temperature as the cold separator 56 but typically at a lower pressure of between about 1.4 MPa (gauge) (200 psig) and about 6.9 MPa (gauge) (1000 psig) and preferably between about 2.4 MPa (gauge) (350 psig) and about 3.8 MPa (gauge) (550 psig). A flashed aqueous stream may be removed from a boot in the cold flash drum 78.

In an embodiment, the hot flash vapor stream may be cooled in the cooler 75 and fed to the cold flash drum 78 to be flashed with the cold liquid stream in the cold bottoms line 60. In an aspect, the cold bottoms line 60 may be joined by the hot flash overhead line 74 and receive the cooled hot flash vapor stream in which the cold bottoms line 60 delivers both streams, the cooled, hot flash vapor stream and the cold liquid stream, to the cold flash drum 78. The cold flash drum 78 may be in downstream communication with the hot flash overhead line 74 of the hot flash stripper 72. The cold flash vapor stream in the cold flash overhead line 80 is rich in hydrogen which may be recovered in a hydrogen recovery section.

The fractionation section 16 may include the stripping column 90 and a fractionation column 110. The stripping column 90 may be in downstream communication the hot bottoms line 54, the hot flash bottoms line 76, the cold bottoms line 60 and/or the cold flash bottoms line 82. In an aspect, the stripping column 90 may be a vessel that contains a cold stripping column and a hot stripping column with a wall that isolates each of the stripping columns from the other. The cold flash liquid stream in the cold flash bottoms line 82 may be heated and fed to the stripping column 90 and the hot flash liquid stream in the hot flash bottoms line 76 may be fed to the stripping column 90. The cold flash liquid stream and the hot flash liquid stream are stripped of gases in the stripping column 90 with a stripping media which is an inert gas such as steam from a stripping media line 96 to provide a stripper vapor stream of naphtha, hydrogen, hydrogen sulfide, steam and other gases in a stripper overhead line 98. The stripper vapor stream in the cold stripper overhead line 98 may be condensed and separated in a receiver 102. A stripper net overhead line 104 from the receiver 102 carries a net stripper off gas of LPG, dry gas, hydrogen sulfide and hydrogen. Unstabilized light naphtha from the bottoms of the receiver 102 may be split between a reflux portion refluxed to the top of the stripping column 90 and a liquid stripper overhead stream which may be transported in a condensed stripper overhead line 106 to further recovery or processing of light naphtha. A sour water stream may be collected from a boot of the overhead receiver 102. A stripped stream in the stripped bottoms line 108 may be heated and fed to the product fractionation column 110. The product fractionation column 110 may be in downstream communication with the stripped bottoms line 108 of the stripping column 90.

The stripping column 90 may be operated with overhead pressure of about 0.7 MPa (gauge) (100 psig), preferably no less than about 0.34 MPa (gauge) (50 psig), to no more than about 2.0 MPa (gauge) (290 psig). The temperature in the overhead receiver 102 ranges from about 38° C. (100° F.) to about 66° C. (150° F.) and the pressure is essentially the same as in the overhead of the stripping column 90. The stripping column 90 may be operated with a bottoms temperature between about 160° C. (320° F.) and about 360° C. (680° F.).

The product fractionation column 110 may be in downstream communication with the stripping column 90 for separating stripped hydrocracked streams into product streams. The product fractionation column 110 may fractionate the stripped stream by means of an inert stripping gas stream fed from stripping line 134. A fractionated overhead stream in an overhead line 148 may be condensed and separated in a receiver 150 with a portion of the condensed liquid being refluxed back to the product fractionation column 110. The product streams from the product fractionation column 110 may include a net fractionated overhead stream comprising light naphtha in a net overhead line 126, a heavy naphtha stream in line 128 from a naphtha side cut outlet, a kerosene stream carried in line 130 from a side cut outlet and a diesel stream in diesel line 132 from a side outlet.

An unconverted oil (UCO) stream boiling above the diesel cut point may be taken in a fractionator bottoms line 140 from a bottom of the product fractionation column 110. A portion or all of the UCO stream in the fractionator bottoms line 140 may be purged from the process, recycled to the hydrocracking reactor 40 or forwarded to a second stage hydrocracking reactor (not shown).

Product streams may also be stripped to remove light materials to meet product purity requirements. The product fractionation column 110 may be operated with a bottoms temperature between about 260° C. (500° F.) and about 385° C. (725° F.), preferably at no more than about 380° C. (715° F.), and at an overhead pressure between about 7 kPa (gauge) (1 psig) and about 69 kPa (gauge) (10 psig). A portion of the UCO stream in the fractionator bottoms line 140 may be reboiled and returned to the product fractionation column 110 instead of adding an inert stripping media stream such as steam in line 134 to heat to the product fractionation column 110.

The naphtha boiling fraction present in the hydrocracked stream in the hydrocracked line 48 will have a greater ratio of concentration of alkylbenzene to concentration of benzene than in the naphtha stream in the naphtha feed line 17 due to the transalkylation of benzene to alkylbenzene in the hydrocracking reactor 40. The hydrocracked stream in the hydrocracked line 48 will have a higher ratio of concentration of alkylbenzene to a concentration of benzene than in the hydrocarbon feed stream in the hydrocarbon line 24 due to the transalkylation of benzene to alkylbenzene in the hydrocracking reactor 40

The naphtha produced in or exiting the hydrocracking reactor 40 in the hydrocracked stream in line 48 will be separated in the net fractionated overhead stream in the net overhead line 126, the liquid stripper overhead stream in the condensed stripper overhead line 106 and the heavy naphtha stream in the naphtha side cut line 128. The net fractionated overhead stream in the net overhead line 126 and the liquid stripper overhead stream in the condensed stripper overhead line 106 will comprise the light naphtha having a T5 and a T95 between about 40 and about 80° C., and the heavy naphtha stream in the naphtha side cut line 128 will comprise the heavy naphtha having a T5 and a T95 between about 80 and about 210° C. The net fractionated overhead stream in the net overhead line 126, the liquid stripper overhead stream in the condensed stripper overhead line 106 and the heavy naphtha stream in the naphtha side cut line 128 will have a higher aggregate concentration ratio of alkylbenzene to aggregate concentration of benzene than in the hydrocarbon feed stream in the hydrocarbon feed line 24 and in the naphtha feed stream in the naphtha feed line 17, preferably prior to supplementation with the benzene recycle stream in the recycle line 174, due to the transalkylation of benzene to alkylbenzene in the hydrocracking reactor 40. Consequently, in an alternative embodiment, the naphtha streams in any of the net fractionated overhead stream in the net overhead line 126, the liquid stripper overhead stream in the condensed stripper overhead line 106, and the heavy naphtha stream in the naphtha side cut line 128 may be transported to the gasoline pool without fear of jeopardizing the benzene specification and instead advantageously reducing the benzene concentration in the gasoline pool.

In a further embodiment, naphtha may be further split in an optional naphtha splitter column 160. Light naphtha from the net fractionated overhead stream in the net overhead line 126 may be mixed with light naphtha from the liquid stripper overhead stream in the condensed stripper overhead line 106 and fed a naphtha splitter column 160. The naphtha splitter column 160 may optionally be preceded by a debutanizer column (not shown).

The naphtha splitter column 160 separates the naphtha stream into a light naphtha stream comprising compounds having five to six carbon atoms and a heavy naphtha stream comprising compounds having more than seven carbon atoms. A fractionated overhead stream in an overhead line 162 may be condensed and separated in a receiver 164 with a portion of the condensed liquid being refluxed back to the naphtha splitter column 160. The net splitter liquid stream comprises the light naphtha stream in a net splitter liquid line 166. LPG may be taken in the net splitter overhead vapor stream in a net splitter gas line 168. A bottoms stream in a splitter bottoms line 170 may be taken and a portion reboiled and returned to the naphtha splitter column 160 while a net splitter bottom stream is taken in the net splitter bottoms line 172 comprising the heavy naphtha stream. If a naphtha splitter column 160 is used, the product fractionation column 110 may not take a heavy naphtha stream in line 128, so that the naphtha stream in line 126 is a full range naphtha. The naphtha splitter column may generally operate at a condenser pressure in the range of about 34 kPag (5 psig) to about 104 kPag (15 psig) and accordance with one embodiment operated at a condenser pressure of about 55 kPag (8 psig) about 85 kPag (12 psig). The bottoms temperature of the naphtha splitter column may be about 85° C. to about 140° C. It is also contemplated that the naphtha splitter column may provide a heart-cut from the side of the column rich in benzene that would be preferably recycled in the recycle line 174.

The light naphtha stream will comprise the predominance of the benzene separated from the hydrocracked stream in the hydrocracked line 48. All or a portion of the light naphtha in the net splitter liquid stream in the net splitter liquid line 166 may be recycled back in a recycle line 174 to supplement the naphtha feed stream in the naphtha feed line 17. A remaining portion of the net splitter liquid stream in the naphtha product line 176 may be fed to a gasoline pool with a lower concentration of benzene than in the fresh naphtha stream in the fresh line 8.

If a naphtha splitter column 160 is used, the naphtha produced in or exiting the hydrocracking reactor 40 in the hydrocracked stream in line 48 will be separated in the light naphtha stream in a net splitter liquid line 166 and the heavy naphtha stream in the net splitter bottom stream in the net splitter bottoms line 172. The light naphtha stream in a net splitter liquid line 166 will comprise the light naphtha having a T5 and a T95 between about 40 and about 80° C., and the heavy naphtha stream in the net splitter bottoms line 172 will comprise the heavy naphtha having a T5 and a T95 between about 80 and about 210° C. The light naphtha stream in a net splitter liquid line 166 and the heavy naphtha stream in the net splitter bottom stream in the net splitter bottoms line 172 will have a higher ratio of aggregate concentration of alkylbenzene to aggregate concentration of benzene than in the hydrocarbon feed stream in the hydrocarbon feed line 24 and in the naphtha feed stream in the naphtha feed line 17, preferably prior to supplementation with the benzene recycle stream in the recycle line 174, due to the transalkylation of benzene to alkylbenzene in the hydrocracking reactor 40. Consequently, in an alternative embodiment, the remaining portion of the net splitter liquid stream in the naphtha product line 176 or the light naphtha stream in a net splitter liquid line 166, if no recycle is taken, and the heavy naphtha stream in the net splitter bottom stream in the net splitter bottoms line 172 may be taken to the gasoline pool without fear of jeopardizing the benzene specification and instead reducing the benzene concentration in the gasoline pool.

Alternatively, the naphtha fraction stream and the distillate fraction stream may be fed to the hydrocracking reactor 40 separately. Most sources of naphtha for naphtha stream 17 will have low nitrogen and sulfur contents and not only do not need to be hydrotreated but would actually be degraded by hydrotreating through hydrogenation of aromatics. For this reason, it may be advantageous to add the naphtha stream in an alternative naphtha feed line 17′.

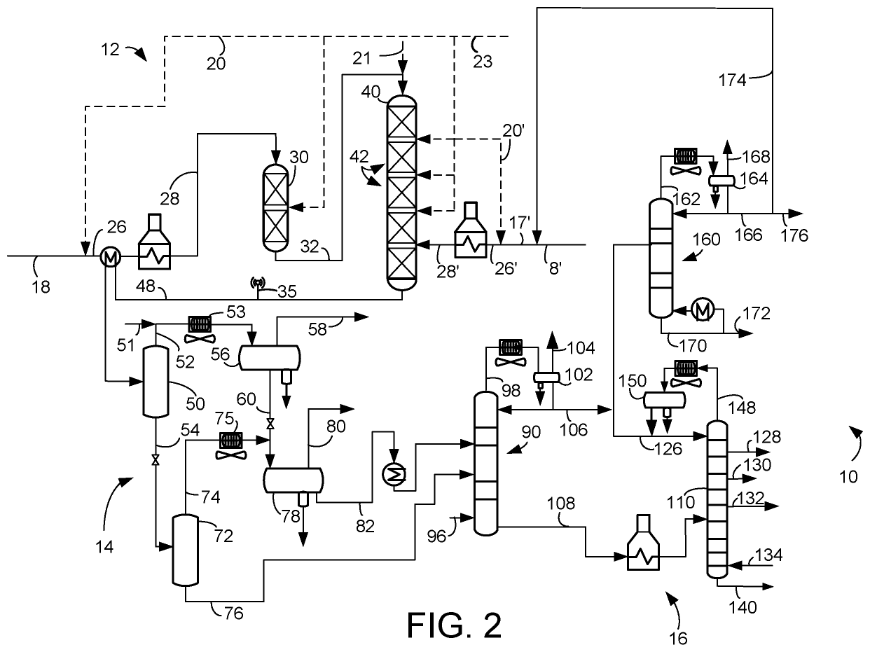

FIG. 2 shows an embodiment in which the naphtha stream is fed toward the later part of the hydrocracking reactor 40. At the front end, a distillate feed line 18 is fed as a hydrocarbon feed stream to the hydrocracking reactor 40. The hydrogen stream in the hydrogen line 20 may split off from a hydrocracking hydrogen line 23. The hydrogen stream in line 20 may be a hydrotreating hydrogen stream. The hydrotreating hydrogen stream may join the hydrocarbon stream in the distillate line 18 to provide a hydrogen-mixed, hydrocarbon feed stream in a hydrocarbon feed line 26. The hydrogen-mixed, hydrocarbon feed stream in the hydrocarbon feed line 26 may be heated by heat exchange with a hydrocracked stream in a hydrocracked line 48 and in a fired heater. The heated hydrocarbon feed stream in line 28 may be fed to the hydrotreating reactor 30 as explained with regard to FIG. 1.

The naphtha fraction stream in an alternative naphtha feed line 17′ may comprise a naphtha recycle stream in a recycle line 174 supplementing a fresh naphtha stream in a fresh line 8′. An alternative hydrogen stream in alternative hydrogen line 20′ taken from the hydrocracking hydrogen line 23 may join the naphtha fraction stream in the alternative naphtha feed line 17′ to provide an alternative hydrogen-mixed, hydrocarbon feed stream in an alternative hydrocarbon feed line 26′. The hydrogen-mixed, hydrocarbon feed stream in the alternative hydrocarbon feed line 26′ may be heated in a fired heater. The heated hydrocarbon feed stream in alternative hydrocarbon feed line 28′ may be fed to the hydrocracking reactor to an interbed location between hydrocracking catalyst beds 42. This naphtha feed location may be preferable to preserve aromatics by avoiding long residence time in the reactor that may lead to hydrogenation of the naphtha-range aromatics. The alternative naphtha feed location may be located between the last two catalyst beds 42 in the hydrocracking reactor 40. Everything else in FIG. 2 is the same as in FIG. 1.

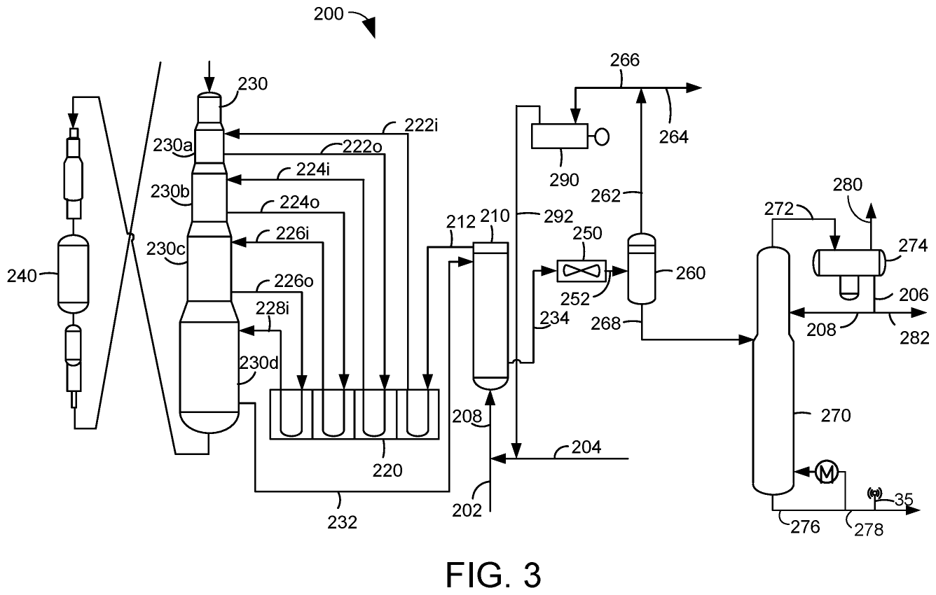

FIG. 3 provides an additional embodiment of a reforming unit 200 for providing a fresh naphtha stream in the fresh line 8 of FIG. 1 or the fresh line 8′ of FIG. 2. The reforming unit 200 comprises a reforming reactor vessel 230, an interzone heater 220, a combined feed exchanger 210, a separator 260, a debutanizer column 270, and a compressor 280. As shown in FIG. 2, a naphtha boiling range feed stream in line 202 is provided to the reforming unit 200. Advantageously, the naphtha boiling range stream can include benzene precursors such as methylcyclopentane and cyclohexane which typically reform to benzene because the later novel use of the hydrocracking reactor described in FIGS. 1 and 2 will convert the benzene to alkylaromatics that are more acceptable for gasoline blending. The naphtha boiling range stream may contain at least about 5 wt % of methylcyclopentane and cyclohexane and suitably at least about 10 wt % methylcyclopentane and cyclohexane in aggregate.

As shown, the naphtha boiling range feed stream in line 202 may be mixed with a reforming hydrogen stream in line 204 supplemented with a recycle hydrogen stream in line 292 to provide a combined feed stream in line 208. The reforming reaction is an endothermic process, so to maintain the reaction, the reforming reactor vessel 230 may comprise an interzone heater 220.

The combined feed stream in line 208 may be heat exchanged with a reformate effluent stream in line 232 in the combined feed exchanger 210 to preheat the combined feed stream in line 208. The preheated feed stream in line 212 may be passed to the reforming reactor 230 of the reforming zone 200. As shown, the reforming reactor vessel 230 may comprise a plurality of reactors 230a, 230b, 230c, and 230d. The reactors may be stacked one on top of another for a stacked reactor configuration to form a compact unit that minimizes plot area requirements. Each of the plurality of reaction zones may be adaptable to contain one or more beds of a reforming catalyst. Each of the plurality of reactors is in fluid communication with an interzone heater 220 to heat the feed stream to the plurality of reactions to a predetermined temperature. Although not shown in FIG. 2, the reactor vessel 230 may comprise single reactor having a fixed bed configuration for the reforming catalyst. In another aspect, the reforming unit 200 comprises a regenerator 240 for continuous regeneration of the spent catalyst by combustion. The regenerator 240 in fluid communication with the reforming reactor vessel 230 may be provided for continuous regeneration of the spent catalyst which is sent back to the reactors after regeneration.

The naphtha boiling range feed may be reformed in the reforming reactor vessel 230 of the reforming unit 200 in the presence of the hydrogen stream and the reforming catalyst to provide a reformate effluent stream. The reactors of the reforming reactor vessel 230 may be operated with a feed inlet temperature from about 450° C. to about 540° C. In the reactors, reforming reactions take place. The primary reforming reactions convert paraffins and naphthenes of the naphtha boiling range feed through dehydrogenation and cyclization to aromatics. The dehydrogenation of paraffins may yield olefins, and the dehydrocyclization of paraffins and olefins may yield aromatics. Particularly, the dehydrogenation and dehydrocyclization of methylcylopentane and cyclohexane yield benzene.

As shown, the preheated feed stream in line 212 may be passed to the interzone heater 220 to provide a first heated feed stream in line 222i which may be passed to the first reaction zone 230a. The first reaction zone effluent in line 222o may be passed to the interzone heater 220 to provide a second heated feed stream in line 224i. The second heated feed stream in line 224i is passed to the second reaction zone 230b. The second reaction zone effluent in line 224o is passed to the interzone heater 220 to provide a third heated feed stream in line 226i. The third heated feed stream in line 226i is passed to the third reaction zone 230c. The third reaction zone effluent in line 226o is passed to the interzone heater 220 to provide a fourth heated feed stream in line 228i. The fourth heated feed stream in line 228i is passed to the fourth reaction zone 230d. Thereafter, a reformate effluent stream in line 232 from the fourth reaction zone may be removed and passed to the combined feed exchanger 210 to preheat the combined feed stream. Although, the reforming unit 200 comprises four reaction zones as shown in FIG. 2, reforming unit 200 may comprise more or less reaction zones depending upon the naphtha boiling range feed to provide the reformate effluent stream.

Reforming catalysts generally comprise a metal on a support. The support can include a porous material, such as an inorganic oxide or a molecular sieve, and a binder. Inorganic oxides used for support include, but are not limited to, alumina, magnesia, titania, zirconia, chromia, zinc oxide, thoria, boria, ceramic, porcelain, bauxite, silica, silica-alumina, silicon carbide, clays, crystalline zeolitic aluminasilicates, and mixtures thereof. Reforming catalysts may comprise one or more Group VIII noble metals. In an exemplary embodiment, the reforming catalyst may comprise one or more of a noble metal selected from platinum, palladium, rhodium, ruthenium, osmium, and iridium. The catalyst can also include a promoter element from Group IIIA or Group IVA. These metals include gallium, germanium, indium, tin, thallium and lead.

As shown, the reformate effluent stream in line 232 may be passed to the combined feed exchanger 210 to provide a heat exchanged reformate effluent stream in line 234. The reformate effluent stream in line 234 may be further cooled in a cooler 250 and passed to the separator 260 in line 252. In the separator 260, the reformate effluent stream in line 234 may be separated to provide a reformate vapor stream in line 262 and a reformate liquid stream in line 268. The reformate vapor stream in line 262 may be split and a recycle vapor stream taken in line 266 and compressed in a recycle compressor 290 to provide the recycle hydrogen stream in line 292. At least a portion of the reformate vapor stream in line 264 may be taken from the reformate vapor stream in line 262 to supply hydrogen needs inside and outside of the reforming unit 200.

At least a portion of the reformate effluent stream may be passed to a debutanizer column 270 of the reforming unit 200 to separate gases from naphtha. In the debutanizer column 270, the reformate liquid stream in line 268 is fractionated to provide an overhead vapor stream in line 272. The overhead vapor stream in line 272 may be passed to a receiver 274 of the debutanizer column 270. In the receiver 274, the overhead vapor stream in line 272 may be separated into the debutanizer column net vapor stream in line 280 comprising dry gas and a receiver liquid stream in line 206. From the receiver liquid stream, a net overhead liquid stream is recovered comprising LPG in a net receiver liquid line 282 comprising LPG while another portion of the receiver liquid stream may be recycled to the debutanizer column 270 as a reflux stream in line 208. The debutanizer bottoms stream in the debutanizer bottoms line 276 may be taken and a portion reboiled and returned to the debutanizer column 270 while a net debutanizer bottom stream is taken in the net debutanizer bottoms line 278 comprising the debutanized naphtha stream which is rich in benzene.

All or a portion of the net debutanizer bottoms stream is naphtha rich in benzene which cannot be blended in the gasoline pool without lowering its octane value. However, feeding this naphtha stream to the hydrocracking reactor 40 via the fresh line 8 in FIG. 1 or the fresh line 8′ in FIG. 2 will transalkylate the benzene to alkylaromatics. The resulting alkylaromatics may blended into the gasoline pool to increase the octane rating of the gasoline pool without negatively impacting compliance with the benzene specification of the gasoline pool.

Network devices 35 can comprise sensors in communication with various streams in lines in FIG. 1 for determining compositions and/or conditions of the stream therein and a transmitter for transmitting a signal or data constituting the measurement to an appropriate receiver. The network devices 35 may be in direct communication, indirect communication, upstream communication and/or downstream communication with the streams in the lines in FIG. 1. The network device 35 may be in a line transporting a stream derived from or fed to a vessel in FIG. 1. For example, network devices 35 with sensors and transmitters may be provided on the hydrocracked line 48 from the hydrocracking reactor 40 to measure a composition and/or condition of the hydrocracked stream therein and transmit a signal constituting the measurement to an appropriate receiver. The sensor may include a temperature gauge, a pressure gauge, a molecular weight analyzer, a specific gravity analyzer, a flow meter, a gas chromatograph, an x-ray diffractometer or any other such sensing or measuring device.

### Example

Hydrocracking was conducted with atmospheric overhead naphtha feed at the following conditions 377° C. (710° F.), LHSV 0.6 hr⁻¹, 6.45 MPa (g) (935 psig), and 1055 Nm³/m³ (6250 SCFB) H₂:Oil over a nickel molybdenum impregnated on Y-zeolite catalyst. The hydrocracked naphtha composition is shown in Table 1 at two weight checks which were averaged for calculations. The approach to equilibrium is 0.1056.

TABLE 1

| | | | Average of |
|---|---|---|---|
| Aromatic | 1^{st} Weight | 2^{nd} Weight | 2 Weight |
| Component | Check, mol % | Check, mol % | Checks, mol % |
| Benzene | 0.2372 | 0.2552 | 0.2463 |
| Toluene | 0.3749 | 0.3576 | 0.3662 |
| Xylenes | 0.2894 | 0.2899 | 0.2897 |
| Trimethyl Benzene | 0.0953 | 0.0900 | 0.0926 |
| Tetramethyl Benzene | 0.0031 | 0.0074 | 0.0053 |
| Methyl/Phenyl | 1.252144 | 1.236759 | 1.244397 |
| ratio for all A6-A10 | | | |

Equilibrium Compositions

| | | | |
|---|---|---|---|
| Benzene | 0.184917 | 0.188964 | 0.186947 |
| Toluene | 0.448001 | 0.451599 | 0.449815 |
| Xylenes | 0.291804 | 0.287202 | 0.289497 |
| Trimethyl Benzene | 0.06866 | 0.065957 | 0.067294 |
| Tetramethyl Benzene | 0.007343 | 0.007017 | 0.007179 |
| Methyl/Phenyl | 1.266961 | 1.25194 | 1.259406 |
| ratio of Equilibrium | | | |

Delta from Equilibrium

| | | | |
|---|---|---|---|
| Benzene | 0.0523 | 0.0662 | 0.0593 |
| Toluene | −0.0731 | −0.0940 | −0.0836 |
| Xylenes | −0.0024 | 0.0027 | 0.0002 |
| Trimethyl Benzene | 0.0266 | 0.0240 | 0.0253 |
| Tetramethyl Benzene | −0.0042 | 0.0003 | −0.0019 |
| Overall Distance | 0.0939 | 0.1175 | 0.1056 |
| from Equilibrium | | | |

We calculated the distance from equilibrium that would occur if we added 25.5 wt % toluene, which acts similar to benzene, and 9.1 wt-% 1,3,5 trimethylbenzene to the hydrocracking feed and assumed the toluene and trimethylbenzene were inert by calculating the distance from equilibrium exhibited by the hydrocracked naphtha produced when toluene and trimethylbenzene were not present in the hydrocracking feed and adding the toluene and trimethylbenzene to the hydrocracked product. The expected distance from equilibrium was calculated to be 0.2974. Table 2 shows the calculation of the distance from equilibrium that would result if the added toluene and trimethylbenzene did not transalkylate.

TABLE 2

| | | Hydrocracked |
|---|---|---|
| | | naphtha product |
| | | if no naphtha |
| | Naphtha Feed, | transalkylation |
| Aromatic | mol % of naphtha | occurred mol % of |
| Component | aromatics | naphtha aromatics |
| Benzene | 0.0000 | 0.0992 |
| Toluene | 0.7852 | 0.6164 |
| Xylenes | 0.0000 | 0.1167 |
| Trimethyl Benzene | 0.2148 | 0.1656 |
| Tetramethyl Benzene | 0.0000 | 0.0021 |
| Methyl/Phenyl | 1.429611115 | 1.355014289 |
| ratio for all A6-A10 | | |

Transalkylation Equilibrium Concentrations

| | | |
|---|---|---|
| Benzene | 0.142685143 | 0.159461933 |
| Toluene | 0.405349098 | 0.423513101 |
| Xylenes | 0.338565136 | 0.320325984 |
| Trimethyl Benzene | 0.102489395 | 0.087703313 |
| Tetramethyl Benzene | 0.011490442 | 0.009634575 |
| Methyl/Phenyl | 1.435909322 | 1.365813307 |
| ratio of Equilibrium | | |

Difference of actual concentration from equilibrium concentration

| | | |
|---|---|---|
| Benzene | −0.1427 | −0.0603 |
| Toluene | 0.3798 | 0.1929 |
| Xylenes | −0.3386 | −0.2037 |
| Trimethyl Benzene | 0.1123 | 0.0779 |
| Tetramethyl Benzene | −0.0115 | −0.0075 |
| Overall Distance | 0.5404 | 0.2974 |
| from Equilibrium | | |

However, in the actual experiment, we discovered that the added benzene and the alkylated aromatics were not inert but transalkylated. The distance from equilibrium decreased in the hydrocracked product from the expected calculated distance in each test in which the toluene and trimethylbenzene were actually blended with the hydrocracking feed and hydrocracked.

TABLE 3

| | Hydrocracked | Hydrocracked | Hydrocracked |
|---|---|---|---|
| Aromatic | Naphtha 1, | Naphtha 2, | Naphtha 3, |
| Component | mol % | mol % | mol % |
| Temperature, | 376.6 (709.8) | 381.2 (718.2) | 384.8 (724.6) |
| ° C., (° F.) | | | |
| Distillate | 51.3 | 64.3 | 67.1 |
| conversion | | | |
| to <193° C. | | | |
| (380° F.) | | | |
| Benzene | 0.1112 | 0.1388 | 0.1410 |
| Toluene | 0.5774 | 0.5401 | 0.5318 |
| Xylenes | 0.1833 | 0.2165 | 0.2244 |
| Trimethyl Benzene | 0.1195 | 0.0960 | 0.0941 |
| Tetramethyl Benzene | 0.0085 | 0.0086 | 0.0087 |
| Methyl/Phenyl | 1.336706208 | 1.295552144 | 1.297615091 |
| ratio for | | | |
| all A6-A10 | | | |

Equilibrium Compositions

| | | | |
|---|---|---|---|
| Benzene | 0.163791877 | 0.17383894 | 0.173324844 |
| Toluene | 0.427922205 | 0.437755225 | 0.437265003 |
| Xylenes | 0.31535961 | 0.304316639 | 0.304893941 |
| Trimethyl Benzene | 0.084195612 | 0.076494977 | 0.076874782 |
| Tetramethyl Benzene | 0.009208237 | 0.008282261 | 0.008327745 |
| Methyl/Phenyl | 1.34841391 | 1.309002477 | 1.31098821 |
| ratio of | | | |
| Equilibrium | | | |

Delta from Equilibrium

| | | | |
|---|---|---|---|
| Benzene | −0.0526 | −0.0350 | −0.0323 |
| Toluene | 0.1495 | 0.1023 | 0.0945 |
| Xylenes | −0.1322 | −0.0878 | −0.0805 |
| Trimethyl Benzene | 0.0353 | 0.0195 | 0.0172 |
| Tetramethyl Benzene | −0.0007 | 0.0003 | 0.0003 |
| Overall Distance | 0.2094 | 0.1406 | 0.1294 |
| from Equilibrium | | | |

All of the actual overall distances from equilibrium were well under the expected calculated distance from equilibrium of 0.2974 had the toluene and the trimethyl benzene been inert. Hence, by analogy the conversion of toluene and trimethylbenzene which were made to be above transalkylation equilibrium by addition to the hydrocracking feed in the Example represent benzene in the subject process which will also decrease in concentration due to being above transalkylation equilibrium by receiving alkyl groups from higher alkylbenzenes with the distance from equilibrium diminishing with increasing reactor temperature.

### Specific Embodiments

While the following is described in conjunction with specific embodiments, it will be understood that this description is intended to illustrate and not limit the scope of the preceding description and the appended claims.

A first embodiment of the disclosure is a process for converting benzene to alkylbenzene comprising reacting a hydrocarbon feed stream comprising a naphtha fraction including at least 5 wt % benzene, alkylated aromatic compounds and a distillate fraction boiling in the range of at least about 193° C. (380° F.) over a hydrocracking catalyst in the presence of hydrogen to hydrocrack the distillate fraction and transalkylate the benzene to alkylbenzene to produce a hydrocracked stream comprising a higher concentration of alkylbenzene and a lower concentration of distillate boiling in the range of at least about 193° C. (380° F.) than the hydrocarbon feed stream. An embodiment of the disclosure is one, any or all of prior embodiments in this paragraph up through the first embodiment in this paragraph further comprising a hydrocracking pressure of about 4.83 MPag (700 psig) to about 10.34 MPag (1500 psig). An embodiment of the disclosure is one, any or all of prior embodiments in this paragraph up through the first embodiment in this paragraph wherein at least about 30 wt % of the hydrocarbon feed stream boils in the range of at least about 193° C. (380° F.). An embodiment of the disclosure is one, any or all of prior embodiments in this paragraph up through the first embodiment in this paragraph further comprising converting at least about 5 wt % of the distillate fraction boiling in the range of at least about 193° C. (380° F.) to material boiling below about 193° C. (380° F.). An embodiment of the disclosure is one, any or all of prior embodiments in this paragraph up through the first embodiment in this paragraph wherein the hydrocarbon feed stream comprises at least 30 wppm nitrogen. An embodiment of the disclosure is one, any or all of prior embodiments in this paragraph up through the first embodiment in this paragraph further comprising separating benzene from the hydrocracked stream and recycling the benzene back to the hydrocracking step. An embodiment of the disclosure is one, any or all of prior embodiments in this paragraph up through the first embodiment in this paragraph further comprising separating a naphtha stream from the hydrocracked stream, the naphtha stream having a higher concentration of alkylbenzene than naphtha in the hydrocarbon feed stream. An embodiment of the disclosure is one, any or all of prior embodiments in this paragraph up through the first embodiment in this paragraph further comprising feeding the naphtha stream with a higher concentration of alkylbenzene into a gasoline pool. An embodiment of the disclosure is one, any or all of prior embodiments in this paragraph up through the first embodiment in this paragraph further comprising feeding a stream of benzene precursors to reform the benzene precursors to benzene over a reforming catalyst to produce a reformate stream comprising benzene and hydrocracking at least a portion of the reformate stream in the hydrocarbon feed stream. An embodiment of the disclosure is one, any or all of prior embodiments in this paragraph up through the first embodiment in this paragraph, further comprising at least one of sensing at least one parameter of the process; and generating or transmitting a signal or data from the sensing.

A second embodiment of the disclosure is a process for converting benzene to alkylbenzene comprising reforming a stream of benzene precursors over a reforming catalyst to benzene to produce a reformate stream comprising benzene; reacting the reformate stream comprising benzene and alkylated aromatic compounds in a hydrocracking reactor over a hydrocracking catalyst in the presence of hydrogen to transalkylate the benzene to alkylbenzene and produce a hydrocracked stream comprising a higher concentration of alkylbenzene than the reformate stream. An embodiment of the disclosure is one, any or all of prior embodiments in this paragraph up through the second embodiment in this paragraph further comprising a hydrocracking pressure of at least 3.5 MPa (gauge) (500 psig). An embodiment of the disclosure is one, any or all of prior embodiments in this paragraph up through the second embodiment in this paragraph further comprising hydrocracking a hydrocarbon feed stream comprising distillate boiling in the range of at least 193° C. (380° F.) with the reformate stream and the hydrocracked stream comprises a lower concentration of distillate boiling in the range of at least 193° C. (380° F.) than the hydrocarbon feed stream. An embodiment of the disclosure is one, any or all of prior embodiments in this paragraph up through the second embodiment in this paragraph wherein at least 5 wt % of the hydrocarbon feed stream boiling in the range of at least 193° C. (380° F.) stream is converted to material boiling below 193° C. (380° F.). An embodiment of the disclosure is one, any or all of prior embodiments in this paragraph up through the second embodiment in this paragraph wherein the hydrocracking feed stream comprises at least 0.1 wt-% sulfur. An embodiment of the disclosure is one, any or all of prior embodiments in this paragraph up through the second embodiment in this paragraph further comprising separating benzene from the hydrocracked stream and recycling the benzene back to the hydrocracking reactor. An embodiment of the disclosure is one, any or all of prior embodiments in this paragraph up through the second embodiment in this paragraph further comprising separating a naphtha stream from the hydrocracked stream, the naphtha stream having a higher concentration of alkylbenzene than the naphtha fraction in the hydrocracking feed stream. An embodiment of the disclosure is one, any or all of prior embodiments in this paragraph up through the second embodiment in this paragraph further comprising feeding the naphtha stream with a higher concentration of alkylbenzene into a gasoline pool.

A third embodiment of the disclosure is a process for converting benzene to alkylbenzene comprising reacting a hydrocarbon feed stream comprising a naphtha fraction including at least 5 wt % benzene, alkylated aromatic compounds and a distillate fraction boiling in the range of at least 199° C. (390° F.) over a hydrocracking catalyst in the presence of hydrogen in a hydrocracking reactor to hydrocrack the distillate fraction and transalkylate the benzene to alkylbenzene to produce a hydrocracked stream comprising a higher concentration of alkylbenzene and a lower concentration of distillate boiling in the range of at least 199° C. (390° F.) than the hydrocarbon feed stream. An embodiment of the disclosure is one, any or all of prior embodiments in this paragraph up through the third embodiment in this paragraph wherein at least 30 wt % of the hydrocracking feed stream comprises distillate boiling above 199° C. (390° F.). An embodiment of the disclosure is one, any or all of prior embodiments in this paragraph up through the third embodiment in this paragraph further comprising separating benzene from the hydrocracked stream and recycling the benzene back to the hydrocracking reactor. Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting benzene to alkylbenzene comprising:
reacting a hydrocarbon feed stream comprising a naphtha fraction including at least 5 wt % benzene, alkylated aromatic compounds and a distillate fraction boiling in the range of at least about 193° C. (380° F.) and having a T95 between about 315° C. (600° F.) and about 650° C. (1200° F.) in a hydrocracking reactor over a hydrocracking catalyst under hydrocracking conditions in the presence of hydrogen to hydrocrack said distillate fraction and transalkylate said benzene to alkylbenzene to produce a hydrocracked stream comprising a higher concentration of alkyl benzene and a lower concentration of distillate boiling in the range of at least about 193° C. (380° F.) than said hydrocarbon feed stream.

2. The process of claim 1 wherein the hydrocracking conditions comprise a hydrocracking pressure of about 4.83 MPag (700 psig) to about 10.34 MPag (1500 psig).

3. The process of claim 1 wherein at least about 30 wt % of the hydrocarbon feed stream boils in the range of at least about 193° C. (380° F.).

4. The process of claim 3 further comprising converting at least about 5 wt % of the distillate fraction boiling in the range of at least about 193° C. (380° F.) to material boiling below about 193° C. (380° F.).

5. The process of claim 1 wherein said hydrocarbon feed stream comprises at least 30 wppm nitrogen.

6. The process of claim 1 further comprising separating benzene from said hydrocracked stream and recycling said separated benzene back to said hydrocracking reactor.

7. The process of claim 1 further comprising separating a naphtha stream from said hydrocracked stream, said separated naphtha stream having a higher concentration of alkyl benzene than the naphtha fraction in said hydrocarbon feed stream.

8. The process of claim 7 further comprising feeding said separated naphtha stream with a higher concentration of alkyl benzene into a gasoline pool.

9. The process of claim 1 further comprising feeding a stream of benzene precursors to reform said benzene precursors to benzene over a reforming catalyst to produce a reformate stream comprising benzene and hydrocracking at least a portion of said reformate stream in said hydrocarbon feed stream.

10. The process of claim 1, further comprising at least one of:
sensing at least one parameter of the process; and
generating or transmitting a signal or data from the sensing.

11. A process for converting benzene to alkylbenzene comprising:
reforming a stream of benzene precursors over a reforming catalyst to benzene to produce a reformate stream comprising benzene;
reacting said reformate stream comprising benzene and alkylated aromatic compounds in a hydrocracking reactor over a hydrocracking catalyst under hydrocracking conditions including temperature of 290° C. (550° F.) to about 468° C. (875° F.) in the presence of hydrogen to transalkylate said benzene to alkylbenzene and produce a hydrocracked stream comprising a higher concentration of alkyl benzene than said reformate stream.

12. The process of claim 11 wherein the hydrocracking conditions comprise a hydrocracking pressure of at least 3.5 MPa (gauge) (500 psig).

13. The process of claim 11 further comprising hydrocracking a hydrocarbon feed stream comprising distillate boiling in the range of at least 193° C. (380° F.) with said reformate stream and said hydrocracked stream comprises a lower concentration of distillate boiling in the range of at least 193° C. (380° F.) than said hydrocarbon feed stream.

14. The process of claim 12 wherein at least 5 wt % of the hydrocarbon feed boiling in the range of at least 193° C. (380° F.) stream is converted to material boiling below 193° C. (380° F.).

15. The process of claim 13 wherein said hydrocarbon feed stream comprises at least 0.1 wt-% sulfur.

16. The process of claim 11 further comprising separating benzene from said hydrocracked stream and recycling said separated benzene back to said hydrocracking reactor.

17. The process of claim 11 further comprising separating a naphtha stream from said hydrocracked stream, said separated naphtha stream having a higher concentration of alkyl benzene than the naphtha fraction in said hydrocracking feed stream.

18. The process of claim 16 further comprising feeding said separated naphtha stream with a higher concentration of alkyl benzene into a gasoline pool.

19. A process for converting benzene to alkyl benzene comprising:
reacting a hydrocarbon feed stream comprising a naphtha fraction including at least 5 wt % benzene, alkylated aromatic compounds and a distillate fraction having an initial boiling point of at least 193° C. (380° F.) over a hydrocracking catalyst in the presence of hydrogen in a hydrocracking reactor to hydrocrack said distillate fraction and transalkylate said benzene to alkylbenzene to produce a hydrocracked stream comprising a higher concentration of alkyl benzene and a lower concentration of distillate boiling in the range of at least 193° C. (380° F.) than said hydrocarbon feed stream.

20. The process of claim 18 wherein at least 30 wt % of the hydrocarbon feed stream comprises distillate boiling above 193° C. (380° F.).
